(19) United States
(12) Patent Application Publication
(10) Pub. No.: US 2022/0331007 A1
(43) Pub. Date: Oct. 20, 2022

(54) RADIO-FREQUENCY ABLATION CATHETER AND RADIO-FREQUENCY ABLATION SYSTEM

(71) Applicant: HANGZHOU BRONCUS MEDICAL CO., LTD., Hangzhou (CN)

(72) Inventors: Liming WANG, Hangzhou (CN); Hong XU, Hangzhou (CN); Huazhen ZHOU, Hangzhou (CN)

(73) Assignee: HANGZHOU BRONCUS MEDICAL CO., LTD., Hangzhou (CN)

(21) Appl. No.: 17/853,357

(22) Filed: Jun. 29, 2022

Related U.S. Application Data
(63) Continuation of application No. PCT/CN2020/118645, filed on Sep. 29, 2020.

(30) Foreign Application Priority Data
Dec. 31, 2019 (CN) 201911403420.3

(51) Int. Cl. A61B 18/14

(52) U.S. Cl. CPC A61B 18/1492; A61B 2018/00577

(57) ABSTRACT

Disclose are a radio-frequency ablation catheter and a radio-frequency ablation system. The radio-frequency ablation catheter includes a needle tube portion and a handle portion. A sleeve of the handle portion is mounted around a booster of the handle portion. A puncture tube of the needle tube portion is fixed to an end of the sleeve. An electrode tube and a signal conduit of the needle tube portion are disposed passing through the puncture tube. A plurality of needles are provided on the electrode tube and a plurality of supports are provided on the signal conduit. The plurality of supports correspond to the plurality of needles respectively. A plurality of temperature sensors are provided at ends of the plurality of supports.

### CROSS REFERENCE OF RELATED APPLICATIONS

This application is a continuation of International Patent Application No. PCT/CN2020/118645, filed on Sep. 29, 2020, which claims the priority of Chinese Patent Application No. 201911403420.3, filed on Dec. 31, 2019, the entire contents of which are hereby incorporated by reference in their entities.

### TECHNICAL FIELD

Embodiments of the present disclosure relate to the field of medical instruments, and particularly to a radio-frequency ablation catheter and a radio-frequency ablation system.

### BACKGROUND

The radio-frequency (RF) ablation technology is widely used in lung surgeries. RF refers to radio frequency, but does not belong to divided bands in radio communication. A main function of RF on organisms is a thermal effect. When a current frequency of the RF reaches a certain value (greater than 100 kHz), charged ions within a tissue will move and thus heat (60° C. to 100° C.) is generated by friction. A frequency commonly used by a radio-frequency ablation device ranges 200 kHz to 500 kHz and an output power thereof ranges from 100 kHz to 400 kHz. An ablation electrode is a core component of a radio-frequency ablation system, because it directly affects a size and a shape of coagulation necrosis. An ideal shape of a coagulation region should be spherical or oblate-sphere. Under the guidance of B-ultrasound or CT, a multi-needle electrode is directly punctured into a tumor tissue. A radio-frequency electrode needle may cause a temperature within the tissue to exceed 60° C. which results in cell death and a necrosis region. If a local temperature of the tissue exceeds 100° C., the coagulation necrosis will occur in a tumor tissue and parenchyma surrounding the organ, and a large spherical coagulation necrosis region may be produced during treatment. A hyperthermia region of 43° C. to 60° C. exists outside the coagulation necrosis region. In this region, cancer cells can be killed while normal cells can be restored.

In a treatment process, the radio-frequency electrode is delivered into a human tissue, and a current is introduced into a lesion through the radio-frequency electrode, resulting in a large amount of heat at the radio-frequency electrode. For example, when a temperature at the lesion reaches 40° C. to 60° C. and remains for a period of time, the ablation surgery at the lesion is completed. However, in a radio-frequency ablation system of the prior art, it cannot determine working state information of the radio-frequency electrode, such as a temperature near the radio-frequency electrode. Therefore, in a surgical process, the progress of the ablation surgery can only be determined and adjusted only based on doctor's experiences, which increases the surgical difficulty and accuracy. Accordingly, how to provide a radio-frequency ablation system to accurately determine whether ablation is completed is an urgent problem to be solved in the art.

### SUMMARY

An objective of the present disclosure is to provide a radio-frequency ablation catheter for a radio-frequency ablation system. A plurality of temperature sensors on a signal conduit are configured to detect temperatures adjacent to a plurality of needles and transmit temperature change ranges to the radio-frequency ablation system, so as to obtain a specific range of a local temperature of a tissue in a surgical process.

Embodiments of the present disclosure provide a radio-frequency ablation catheter for a radio-frequency ablation system, including a needle tube portion and a handle portion, wherein

the handle portion includes a sleeve and a booster, wherein the sleeve is mounted around the booster, the booster is slidably arranged at one end of the sleeve, the booster is provided with a conductive joint, and the conductive joint is configured for connection with an external radio-frequency ablation system;

the needle tube portion includes a puncture tube, an electrode tube and a signal conduit, wherein

the puncture tube is fixed at an another end of the sleeve, the electrode tube is slidably disposed within the puncture tube, one end of the electrode tube is fixed to the conductive joint and an another end of the electrode tube is provided with a plurality of needles, the plurality of needles are configured to transfer a current provided by the conductive joint, the signal conduit is slidably disposed in the puncture tube, the signal conduit is positioned on one side of the electrode tube, and one end of the signal conduit is fixed to the conductive joint;

the signal conduit includes a plurality of supports and a plurality of temperature sensors;

an another end of the signal conduit is provided with the plurality of supports, and the plurality of supports are positioned on first sides of the plurality of needles; and

the plurality of temperature sensors are respectively disposed on the plurality of supports and are electrically connected thereto, and the plurality of temperature sensors are configured to detect temperatures adjacent to the plurality of needles and transmit the temperatures to the radio-frequency ablation system by means of the signal conduit.

The present disclosure further provides a radio-frequency ablation system, including a radio-frequency ablation catheter according to anyone of the above feasible embodiments.

As can be seen from the above solutions, the radio-frequency ablation catheter of the present disclosure includes the needle tube portion and the handle portion. The sleeve on the handle portion is mounted around the booster of the handle portion. One end of the booster is provided with the conductive joint, and the conductive joint is used for connection with the external radio-frequency ablation system. The puncture tube on the needle tube portion is fixed at one end of the sleeve, the electrode tube and the signal conduit of the needle tube portion are fixed on the conductive joint, and the signal conduit is positioned on one side of the electrode tube. The plurality of needles are disposed at the end of the electrode tube, and the plurality of supports are disposed at the end of the signal conduit. Each support is provided with a respective capacitance thermometer. In the radio-frequency ablation catheter of the present disclosure, the signal conduit and the electrode tube are fixed on the conductive joint, the plurality of supports are fixed on first sides of the plurality of needles, and the corresponding capacitance thermometers are disposed on the plurality of supports and configured to detect changes in the temperatures near the needles. The radio-frequency ablation system releases the current into the human tissue through the electrode tube and the needles, and causes a large number of dielectrics such as ions, water and colloidal particles in a human body fluid to move at high speeds with the current under the action of a radio-frequency current at high frequency. Due to differences in sizes, mass charges and moving speeds of the ions, the tissue generates a biological heat effect due to friction of the ions, and thus the local temperature of the tissue increases. The capacitance thermometers on the supports sense the temperatures near the corresponding needles and transmit the temperatures to the radio-frequency ablation system through the signal conduit. The changes of the temperature within the human tissue can be directly displayed on the radio-frequency ablation system. According to the temperature change range measured by the capacitance thermometer, an output current of the radio-frequency ablation system can be controlled. As such, it is reliable to control the current in the surgery.

### BRIEF DESCRIPTION OF THE DRAWINGS

In order to explain embodiments of the present disclosure or technical solutions of the present disclosure more clearly, accompanying drawings required to be used in describing the embodiments or the prior art will be briefly introduced below. Obviously, the accompanying drawings in the following description are only some embodiments of the present disclosure. For those ordinarily skilled in the art, other accompanying drawings may be obtained based on these accompanying drawings without any creative labor.

FIG. 1 is a structural schematic view of a radio-frequency ablation catheter according to a first embodiment of the present disclosure;

FIG. 2 is a partially enlarged schematic view showing a needle tube portion according to the first embodiment of the present disclosure;

FIG. 3 is a structural schematic view a radio-frequency ablation catheter according to a third embodiment of the present disclosure;

FIG. 4 is a partially enlarged schematic view showing a needle tube portion according to the third embodiment of the present disclosure;

FIG. 5 is a structural schematic view of a radio-frequency ablation catheter according to a fourth embodiment of the present disclosure;

FIG. 6 is a partially enlarged schematic view showing a needle tube portion according to the fourth embodiment of the present disclosure;

FIG. 7 is a view illustrating a push-out state of a booster according to the first embodiment of the present disclosure;

FIG. 8 is a perspective view showing needles and supports according to the fourth embodiment of the present disclosure; and

FIG. 9 is a schematic view showing needles and supports at the same latitude according to a fifth embodiment of the present disclosure.

### REFERENCE NUMERALS IN THE FIGURES

1: needle tube portion; 11: puncture tube; 12: electrode tube; 121: needle; 122: metal ball; 13: signal conduit; 131: support; 132: temperature sensor; 2: handle portion; 21: sleeve; 22: booster; 221: conductive joint; 3: fixed ring; 31: through hole; and 4: insulating layer.

### DESCRIPTION OF THE EMBODIMENTS

In order to make objectives, technical solutions, and advantages of embodiments of the present disclosure clearer, the technical solutions in the embodiments of the present disclosure will be further described in detail below with reference to accompanying drawings in the embodiments of the present disclosure. Obviously, the described embodiments are only some, but not all of the embodiments of the present disclosure. Based on the embodiments of the present disclosure, all other embodiments obtained by those ordinarily skilled in the art without any creative labor shall fall within a protective scope of the present disclosure.

In the description of the present disclosure, it should be understood that orientations or position relationships indicated by terms “center”, “longitudinal”, “transverse”, “upper”, “lower”, “front”, “rear”, “left”, “right”, “vertical”, “horizontal”, “top”, “bottom”, “inner”, “outer”, “axial”, “radial” and “circumferential” are based on orientations or position relationships shown in the accompanying drawings, only for the convenience of describing the present disclosure and simplifying the description, rather than indicating or implying that a device or element in question must have a specific orientation and is constructed and operated in a specific orientation, such that they may not be understood as a limitation of the present disclosure.

In the present disclosure, unless otherwise specified and limited, terms “install”, “connect with”, “connect”, “fix” and the like shall be understood in a broad sense, for example, they may be fixed connection, removable connection or integrated; or they may be mechanical connection, electrical connection or communication connection; or they may be direct connection or indirect connection through an intervening medium; or they may be communication inside two elements or interaction relationship between two elements, unless otherwise clearly defined. For those skilled in the art, specific meanings of the above terms in the present disclosure may be understood according to specific situations. The technical solutions of the present disclosure will be described in detail below in combination with specific embodiments. The following specific embodiments may be combined with each other, and the same or similar concepts or processes may be omitted in some embodiments.

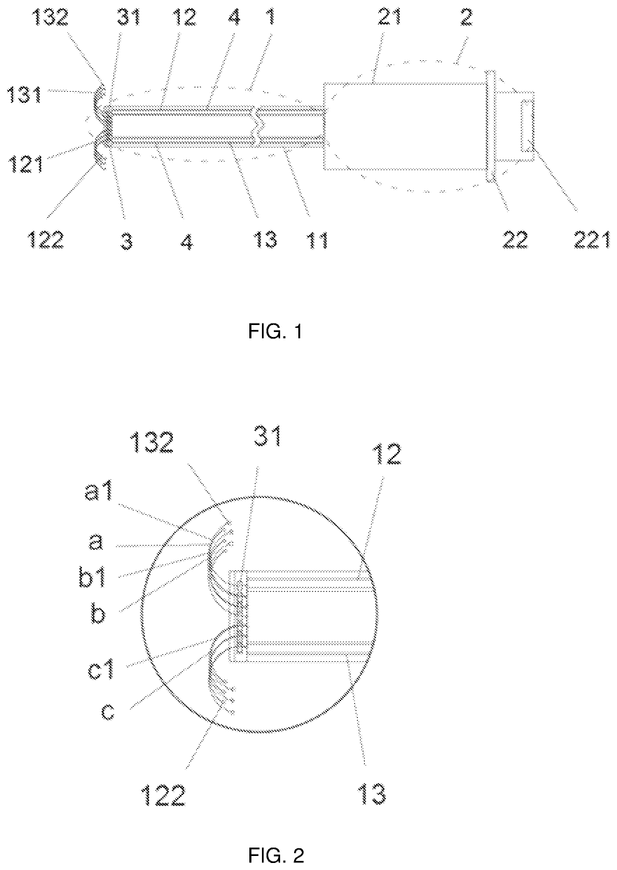

FIG. 1 is a structural schematic view of a radio-frequency ablation catheter according to a first embodiment of the present disclosure.

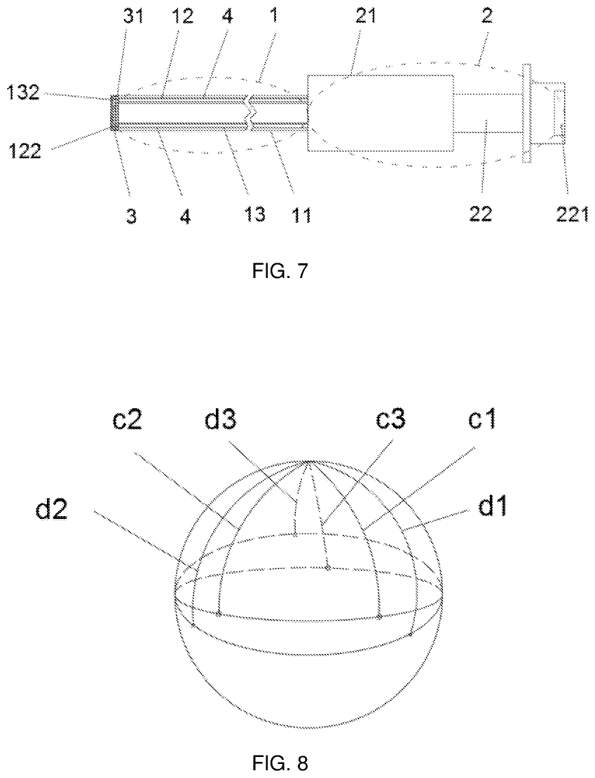

The radio-frequency ablation catheter in this embodiment is applied to a radio-frequency ablation system. The radio-frequency ablation catheter includes a needle tube portion 1 and a handle portion 2. The handle portion 2 includes a sleeve 21 and a booster 22. The sleeve 21 is mounted around one end of the booster 22, and the booster 22 is slidable inside the sleeve 21. A conductive joint 221 is provided at the other end of the booster 22. In this embodiment, four electrode insertion holes (not shown) are provided on the conductive joint 221, and configured for insertion of four electrode terminals on a connector of the radio-frequency ablation system. The needle tube portion 1 includes a puncture tube 11, an electrode tube 12 and a signal conduit 13. Both the electrode tube 12 and the signal conduit 13 are disposed inside the puncture tube 11. The signal conduit 13 is positioned on one side of the puncture tube 11 and is movable in the puncture tube 11. One end of the puncture tube 11 is fixed to an end of the sleeve 21. One end of the electrode tube 12 and one end of the signal conduit 13 are fixed on the conductive joint 221. The electrode tube 12 and the signal conduit 13 pass into the sleeve 21. When the booster 22 is pushed into the sleeve 21, the electrode tube 12 and the signal conduit 13 can be pushed out from the puncture tube 11 by means of an action of the booster 22 (as shown in FIG. 7, which is a view shows push-out states of the electrode tube and the signal conduit from a puncture tube). When the electrode tube and the signal conduit are pushed out from the puncture tube, metal balls and temperature sensors are separately arranged in a staggered manner, and thus frictions between the metal balls and the temperature sensors in a converged state may be reduced. A plurality of needles 121 are provided on the electrode tube 12, and a plurality of supports 131 are provided on the signal conduit 13, wherein the number of the supports 131 is equal to that of the needles 121. The needles 121 on the electrode tube 12 are released in an umbrella shape (see FIG. 2), and the supports 131 are released in an umbrella shape. A current from the radio-frequency ablation system is transferred into a human tissue through the conductive joint 221, the electrode tube 12 and the needles 121. Each support 131 is provided with a temperature sensor 132 fixed at an end thereof. The temperature sensor 132 may be a capacitance thermometer, with a capacitor as a sensing element, a conversion device which converts the measured temperature into a capacitance change is formed as a capacitor with a variable parameter. As the temperature increases, capacitance characteristics decrease gradually. A change in the capacitance characteristics is fed back to the radio-frequency ablation system, and a temperature change range may be seen directly. A large amount of dielectrics such as ions, water and colloidal microparticles in a human body fluid move at high speeds with the current under the action of a radio-frequency current at high frequency transmitted from the needles 121. Due to differences in sizes, mass charges and movement speeds of the ions, frictions of the ions are caused and the tissue generates a biological heat effect, and thus the local temperature of the tissue increases. The capacitance thermometers on the supports 131 next to the needles 121 can be used to detect a temperature inside the tissue. Each capacitance thermometer transmits the detected temperature value to the radio-frequency ablation system through the signal conduit 13, converts the sensed temperature into a displayable output signal, and thus directly showing a temperature range.

As it can be seen from the above contents that the radio-frequency ablation catheter of the present disclosure includes the needle tube portion 1 and the handle portion 2. The sleeve 21 on the handle portion 2 is mounted around the booster 22 of the handle portion 2. One end of the booster 22 is provided with the conductive joint 221, and the conductive joint 221 is used to be connected to the external radio-frequency ablation system. The puncture tube 11 of the needle tube portion 1 is fixed to one end of the sleeve 21, the electrode tube 12 and the signal conduit 13 of the needle tube portion 1 are both fixed on the conductive joint 221, and the signal conduit 13 is positioned on one side of the electrode tube 12. The plurality of needles 121 are provided at the distal end of the electrode tube 12, and the plurality of supports 131 are provided at the distal end of the signal conduit 13. Each support 131 is provided with a respective capacitance thermometer. The signal conduit, the supports and the capacitance thermometers are electrically connected. In the radio-frequency ablation catheter of the present disclosure, the signal conduit 13 and the electrode tube 12 are fixed to the conductive joint 221, the plurality of supports 131 each are fixed on one side of a respective one of the plurality of needles 121, and the capacitance thermometers are correspondingly disposed on the plurality of supports 131 and configured to detect temperature changes near the needles 121. The radio-frequency ablation system releases the current into the human tissue through the electrode tube 12 and the needles 121, and enables a large number of dielectrics such as ions, water and colloidal particles in a human body fluid to move at a high speed under the action of the radio-frequency current at high frequency. Due to differences in sizes, mass charges and movement speeds of the ions, frictions of the ions are caused and the tissue generates a biological heat effect, and thus the local temperature of the tissue increases. The capacitance thermometers on the supports 131 sense temperatures next to the corresponding needles 121, and transmit the temperatures to the radio-frequency ablation system through the signal conduit 13, such that the temperatures in the human tissue can be directly displayed on the radio-frequency ablation system. The output current of the radio-frequency ablation system can be controlled according to the temperature change ranges measured by the capacitance thermometers, such that it is possible to achieve the controllability of the current in the surgery.

As shown in FIG. 2, optionally, in this embodiment, a plurality of needles 121 are provided on the electrode tube 12, a plurality of supports 131 are provided on the signal conduit 13, and the signal conduit 13 is fixed on one side of the electrode tube 12. The number of the needles 121 is equal to that of the supports 131. A respective support 131 is disposed next to each needle 121, and distances and angles between each needle 121 and the corresponding support 131 are identical. For example, a corresponding support a1 is disposed next to the needle a, a corresponding support b1 is disposed next to the needle b, a corresponding support c1 is disposed next to the needle c, each needle 121 is provided with a corresponding support 131. A distance between distal ends of the needle a and the support c1 is 0.2 cm, a distance between a distal end of the needle b and a distal end of the support b1 is 0.2 cm, a distance between a distal end of the needle c and a distal end of the support c1 is 0.2 cm, a distance between the needle a and the needle b and a distance between the needle b and the needle c are both 0.4 cm. a distance between the support a1 and the support b1 and a distance between the support b1 and the support c1 are both 0.4 cm as well. Distances between adjacent needles 121 are constant, and distances between adjacent supports 131 are constant.

Optionally, in this embodiment, each needle 121 is provided with a metal ball 122 welded at the distal end thereof, and a ratio of an outer diameter of each metal ball 122 to a diameter of the corresponding needle 121 is 1.05:1.01. The outer diameter of the metal ball 122 is slightly greater than the diameter of the needle 121. The metal balls are arranged in a staggered manner within the puncture tube, such that the metal balls may be well retracted into the puncture tube. The metal balls, the needles and the electrode tube are electrically connected. The surface area of the metal ball is spherical, which is greater than the interface area between the needle 121 and the human tissue, such that the current release area can be increased. Accordingly, more tissue cells are in contact with the current, and thus increasing the working efficiency of the radio-frequency ablation catheter.

Optionally, in this embodiment, the radio-frequency ablation catheter further includes a fixed ring 3. The fixed ring 3 is snap fitted at a position not far away from an opening of the puncture tube 11. The fixed ring 3 is a circular ring. A plurality of through holes 31 are provided in the circular ring, and the number of these through holes 31 is equal to the sum of the number of needles 121 and the number of the supports 131. The diameter of the through hole 31 is greater than that of the needle 121 and greater than that of the support 131. The plurality of needles 121 and supports 131 extend through the through holes 31 respectively. The diameter of the metal ball 122 and the diameter of the capacitance thermometers are both greater than that of the through hole 31 which facilitates the electrode tube 12 and the signal conduit 13 to extend out, and the positions of the plurality of supports 131 and the plurality of needles 121 can be fixed.

Optionally, in this embodiment, outer peripheries of the electrode tube 12 and the signal conduit 13 are wrapped with insulating layers 4. The insulating layers 4 are made of plastic. Due to the current passing through the electrode tube 12, the signal conduit 13 converts the temperature into an output signal. As such, in order to prevent interference between the electrode tube 12 and the signal conduit 13, the plastic on the outer peripheries of the electrode tube 12 and the signal conduit 13 can prevent signal interference therebetween, thus achieving the function of shielding signals.

A second embodiment is an alternative of the first embodiment, with a difference in that the capacitance thermometer in the first embodiment is replaced with a thermistor. The thermistor is sensitive to the temperature and has different resistance values at different temperatures. The higher the temperature is, the lower the resistance value is. Under the effect of the current, ions and media within the human tissue will move at high speeds and thus heat is generated locally. When the temperature becomes higher and higher, the resistance within of the thermistor located in a local region will decrease with the increase of the temperature. The thermistors, the supports and the signal conduit are electrically connected. When the resistance values of the thermistors change, the change in the resistance values is transmitted to the radio-frequency ablation system by means of the signal conduit 13, and a change range of a local temperature of the tissue is calculated on the radio-frequency ablation system according to the change in the resistance value, such that the temperature can be controlled by controlling the output current of the radio-frequency ablation system.

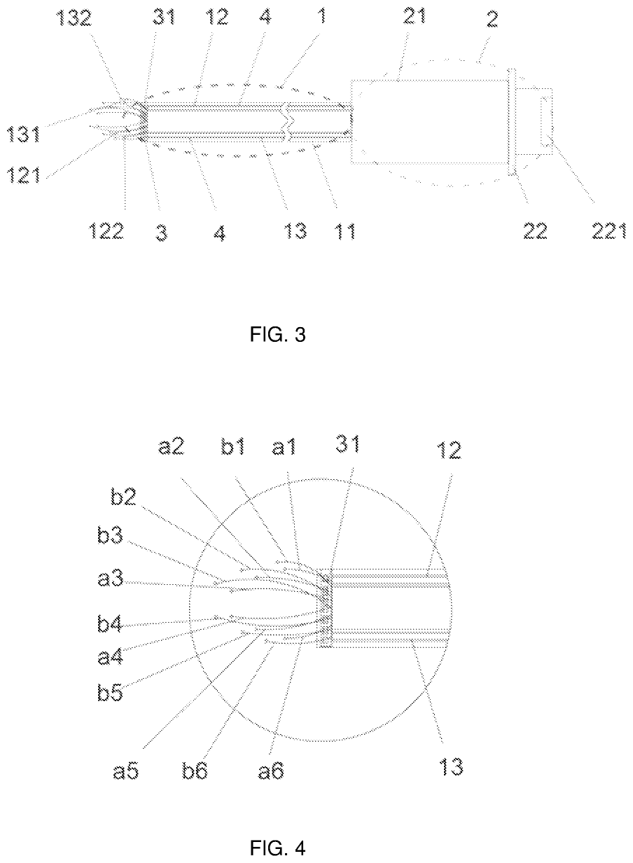

FIG. 3 is a structural schematic view a radio-frequency ablation catheter according to a third embodiment of the present disclosure.

As described in FIG. 3, the third embodiment is a modification of the first embodiment, with a development that the plurality of needles 121 and the plurality of supports 131 are arranged in a flower-radiation shape (see FIG. 4). The lengths of the plurality of needles 121 are different, and the positions of the plurality of needles 121 are distributed symmetrically with respect to a central axis of the electrode tube 12. It is assumed that six needles 121 are disposed on the electrode tube 12, and named as a1, a2, a3, a4, a5 and a6 respectively, wherein the needle a1 and the needle a6 are symmetrical with respect to the central axis of the electrode tube 12, the needle a2 and the needle a5 are symmetrical with respect to the central axis of the electrode tube 12, and the needle a3 and the needle a4 are symmetrical with respect to the central axis of the electrode tube 12. Moreover, the lengths of the needle a1 and the needle a6 are identical, the lengths of the needle a2 and the needle a5 are identical, and the lengths of needle a3 and the needle a4 are identical. The needle a1 and the needle a6 are positioned on outermost sides of all needles 121 and have the shortest length, followed by the needle a2 and the needle a5, the lengths of which are greater than the lengths of the needle a1 and the needle a6. The longest needles are the needle a3 and the needle a4 which are positioned in the middlemost position. According to such a position arrangement, different positions of respective needles 121 make the current flow through different positions. As a result, a region covered by current flows can be enlarged and thus facilitating more human tissues generating heat. The plurality of supports 131 have different lengths, and the positions of the plurality of supports 131 are distributed symmetrically with respect to the central axis of the signal conduit 13. It is assumed that six supports 131 are disposed on the signal conduit 13, and are named as b1, b2, b3, b4, b5 and b6 respectively, wherein the support b1 and the support b6 are symmetrical with respect to the central axis of the signal conduit 13, the support b2 and the support b5 are symmetrical with respect to the central axis of the signal conduit 13, and the support b3 and the support b4 are symmetrical with respect to the central axis of the signal conduit 13. Moreover, the lengths of the support b1 and the support b6 are identical, the lengths of the support b2 and the support b5 are identical, and the lengths of the support b3 and the support b4 are identical. The support b1 and the support b6 are positioned at outermost sides of all supports 131 and have the longest length, followed by the support b2 and the support b5, the lengths of which are greater than the lengths of the support b1 and the support b6. The longest supports are the support b3 and the support b4, which are positioned in the middlemost position. The positions of the supports 131 are set according to the positions of the needles 121. Each support 131 corresponds to a needle 121. In this way, the temperature value generated near the corresponding needle 121 can be particularly measured by the capacitance thermometer on the corresponding support 131. According to the difference in the heat generated by each needle 121, the temperatures measured by the capacitance thermometers are different as well, the resulting temperatures are more targeted, and the resulting feedback data will be different as well. These temperature values are transmitted to the radio-frequency ablation system by means of the signal conduit 13. Accordingly, a specific temperature range may be obtained.

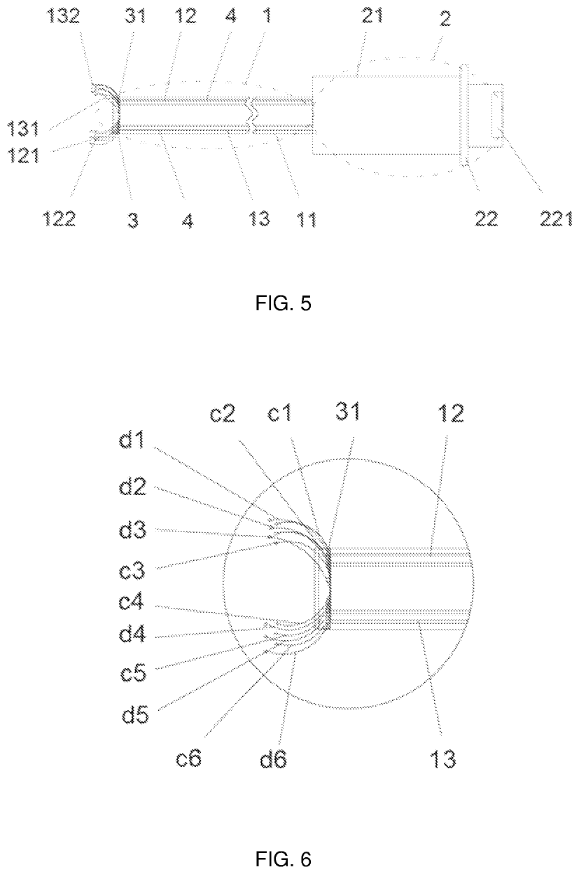

FIG. 5 is a structural schematic view of a radio-frequency ablation catheter according to a fourth embodiment of the present disclosure.

The fourth embodiment shown in FIG. 5 is a modification of the first embodiment, with a development that the plurality of needles 121 and the plurality of supports 131 are distributed in a semi-spherical configuration in space. As shown in FIG. 6, the plurality of needles 121 may be named as c1, c2, c3, c4, c5 and c6 respectively, wherein the needles c1, c2, c3, c4, c5 and c6 are sequentially arranged in a clockwise direction, and have the same length. Since the six needles 121 are distributed in the spherical manner in space, although the six needles 121 are in different horizontal positions, according to the spherical configuration, the corresponding metal balls 122 on the six needles 121 are located at the same latitude, the current covers a wider region from left to right and a larger transverse distribution area, which facilitates contact of more lateral tissue cells. The plurality of supports 131 may be named as d1, d2, d3, d4, d5 and d6 respectively, wherein the supports d1, d2, d3, d4, d5 and d6 are sequentially arranged in a clockwise direction. Each support 131 corresponds to a needle 121, a capacitance thermometer on each support 131 is configured to detect a temperature near the corresponding metal ball 122, and the supports d1, d2, d3, d4, d5 and d6 have the same length. According to a distribution profile of the spherical configuration as shown in FIG. 8, since the six supports 131 are distributed in the spherical configuration in space, although the capacitance thermometers on the supports d1, d2, d3, d4, d5 and d6 are at different horizontal positions in terms of horizontal positions, according to the spherical configuration, the corresponding capacitance thermometers on the six supports 131 are at the same latitude, and each capacitance thermometer transmits the detected temperature near the corresponding metal ball 122 to the radio-frequency ablation system by means of the signal conduit 13. As a result, the corresponding temperature change range may be seen directly on the radio-frequency ablation system. Further, the output current of the radio-frequency ablation system may be adjusted according to the change in the temperature.

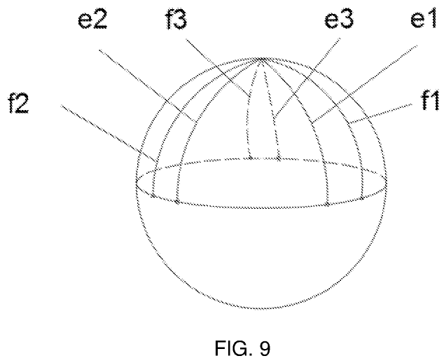

FIG. 9 is a structural schematic view according to a fifth embodiment of the present disclosure, showing the needles and the supports at the same latitude.

The fifth embodiment as shown in FIG. 9 is a modification of the first embodiment, with a development that the plurality of needles 121 and the plurality of supports 131 are distributed in a spherical shape. The plurality of needles may be named as e1, e2 and e3 respectively, wherein the e1, the e2 and the e3 have the same length. Since the three needles 121 are distributed in the spherical space, although the three needles 121 are in different horizontal positions, according to the spherical shape in space, the corresponding metal balls 122 on the three needles 121 are at the same latitude. The plurality of supports 131 may be named as f1, f2 and f3 respectively. Each support 131 corresponds to a needle 121. The capacitance thermometer on each support 131 is configured to detect a temperature near the corresponding metal ball 122, and the supports f1, f2 and f3 have the same length. According to the distribution of the spherical shape in space as shown in FIG. 9, since the three supports 131 are distributed in the spherical shape in space, although the capacitance thermometers on the supports f1, f2 and f3 are in different horizontal positions in terms of horizontal positions, according to the spherical shape in space, the corresponding capacitance thermometers on the three supports 131 are at the same latitude. For this embodiment, the metal balls and the capacitance thermometers are at the same latitude in the spherical space.

In the present disclosure, unless otherwise expressly specified and limited, a first feature “on” or “under” a second feature may mean that the first feature is in direct contact with the second feature, or the first feature is in indirect contact with the second feature through an intervening medium.

Moreover, the first feature “on”, “above” and “over” the second feature may mean that the first feature is directly above or obliquely above the second feature, or only means that a horizontal height of the first feature is higher than that of the second feature. The first feature “under”, “below” and “underneath” the second feature may mean that the first feature is directly below or obliquely below the second feature, or only means that the horizontal height of the first feature is lower than that of the second feature.

In the description of this specification, reference is made to descriptions of terms “one embodiment”, “some embodiments”, “examples”, “specific examples” or “some examples”, which means that specific features, structures, materials or characteristics described in combination with embodiments or examples are included in at least one embodiment or example of the present disclosure. In this specification, schematic expressions of the above terms do not have to be for the same embodiments or examples. Further, the specific features, structures, materials or characteristics described may be combined in a suitable fashion in any one or more embodiments or examples. In addition, those skilled in the art may incorporate and combine different embodiments or examples described in this specification and features of different embodiments or examples, without mutual contradiction.

Finally, it should be noted that the above embodiments are only used to explain the technical solution of the present disclosure and shall not be construed as limitation. Although the present disclosure has been described in detail with reference to the above embodiments, those skilled in the art should understand that it may still modify the technical solutions recorded in the above embodiments or substitute some or all of the technical features equally, without making the essence of the corresponding technical solution deviate from the scope of the technical solution of each embodiment of the present disclosure.

What is claimed is:

1. A radio-frequency ablation catheter for a radio-frequency ablation system, comprising a needle tube portion and a handle portion, wherein
the handle portion comprises a sleeve and a booster, wherein the sleeve is mounted around the booster, the booster is slidably arranged at one end of the sleeve, the booster is provided with a conductive joint, and the conductive joint is configured for connection with an external radio-frequency ablation system;
the needle tube portion comprises a puncture tube, an electrode tube and a signal conduit, wherein
the puncture tube is fixed at an another end of the sleeve, the electrode tube is slidably disposed within the puncture tube, one end of the electrode tube is fixed to the conductive joint and an another end of the electrode tube is provided with a plurality of needles, the plurality of needles are configured to transfer a current provided by the conductive joint, the signal conduit is slidably disposed in the puncture tube, the signal conduit is positioned on one side of the electrode tube, and one end of the signal conduit is fixed to the conductive joint;
the signal conduit comprises a plurality of supports and a plurality of temperature sensors;
an another end of the signal conduit is provided with the plurality of supports, and the plurality of supports are positioned on first sides of the plurality of needles; and
the plurality of temperature sensors are respectively disposed on the plurality of supports and are electrically connected thereto, and the plurality of temperature sensors are configured to detect temperatures adjacent to the plurality of needles and transmit the temperatures to the radio-frequency ablation system by means of the signal conduit.

2. The radio-frequency ablation catheter according to claim 1, wherein the plurality of temperature sensors are respectively positioned at distal ends of the plurality of supports.

3. The radio-frequency ablation catheter according to claim 1, wherein the temperature sensors are capacitance thermometers or thermistors.

4. The radio-frequency ablation catheter according to claim 1, wherein a number of the plurality of supports is equal to a number of the plurality of needles, and wherein the plurality of supports and the plurality of needles are arranged alternately.

5. The radio-frequency ablation catheter according to claim 4, wherein the plurality of supports are respectively disposed next to the plurality of needles, and distances between the plurality of needles and the respective supports are identical.

6. The radio-frequency ablation catheter according to claim 1, wherein the electrode tube further comprises a plurality of metal balls respectively disposed at distal ends of the plurality of needles, wherein
a ratio of an outer diameter of each metal ball to a diameter of a corresponding needle is 1.05:1.01, and the plurality of metal balls are electrically connected to the plurality of needles respectively.

7. The radio-frequency ablation catheter according to claim 1, comprising a fixed ring, wherein the fixed ring is positioned within the puncture tube, and the fixed ring is configured to fix the plurality of supports and the plurality of needles.

8. The radio-frequency ablation catheter according to claim 7, wherein the fixed ring is provided with a plurality of through holes, a number of the plurality of through holes is equal to a sum of a number of the plurality of supports and a number of the plurality of needles, and wherein the plurality of supports and the plurality of needles extend through the plurality of through holes respectively.

9. The radio-frequency ablation catheter according to claim 1, wherein surfaces of the electrode tube and the signal conduit are provided with insulating layers, and the insulating layers are configured to shield a signal.

10. The radio-frequency ablation catheter according to claim 1, wherein the plurality of needles are arranged in a flower-radiation shape, and the plurality of supports are arranged in a flower-radiation shape.

11. The radio-frequency ablation catheter according to claim 10, wherein each needle has a length, and the lengths of the needles located at a middle position are the longest, the lengths of the needles located at outermost ends are the shortest, and the lengths of the plurality of needles are gradually decreased from the middle position towards two ends.

12. The radio-frequency ablation catheter according to claim 11, wherein the plurality of needles are distributed symmetrically with respect to a central axis of the electrode tube, and the lengths of the corresponding needles symmetrical with respect to the central axis of the electrode tube are identical.

13. The radio-frequency ablation catheter according to claim 10, wherein each support has a length, and the lengths of the supports located in a middle position are the longest, the lengths of the supports located at outmost ends are the shortest, and the lengths of the plurality of supports are gradually decreased from the middle position towards two ends.

14. The radio-frequency ablation catheter according to claim 13, wherein the plurality of supports are distributed symmetrically with respect to a central axis of the signal conduit, and the lengths of the corresponding supports symmetrical with respect to the central axis of the signal conduit are identical.

15. The radio-frequency ablation catheter according to claim 6, wherein the plurality of needles are annularly distributed in a spherical shape in space, and the plurality of supports are annularly distributed in a spherical shape in space.

16. The radio-frequency ablation catheter according to claim 15, wherein the metal balls are located at a same latitude on the spherical shape.

17. The radio-frequency ablation catheter according to claim 15, wherein the temperature sensors are located at a same latitude on the spherical space.

18. The radio-frequency ablation catheter according to claim 15, wherein the temperature sensors and the metal balls are located at a same latitude.

19. A radio-frequency ablation system, comprising a radio-frequency ablation catheter according to claim 1.

20. An ablation method using the radio-frequency ablation catheter according to claim 1, comprising:
delivering the radio-frequency ablation catheter in a human body to a tissue to be ablated;
supplying a current to the plurality of needles and ablating the tissue by the plurality of needles;
acquiring temperatures of the tissue adjacent to the plurality of needles by the plurality of temperature sensors on the plurality of supports respectively; and
controlling the current according to the temperatures measured by the plurality of temperature sensors.
